(19) United States
(12) Patent Application Publication
(10) Pub. No.: US 2017/0281313 A1
(43) Pub. Date: Oct. 5, 2017

(54) METHOD AND SYSTEM FOR PROVIDING REMOTE TEETH ALIGNMENT SERVICES AND COMPUTER-READABLE MEDIUM ON WHICH THE METHOD IS RECORDED

(71) Applicant: eClear International Co., Ltd., Los Angeles, CA (US)

(72) Inventor: TAEWEON KIM, Seoul (KR)

(21) Appl. No.: 15/090,720

(22) Filed: Apr. 5, 2016

(51) Int. Cl. A61C 7/00; G06K 9/00; G06F 19/00; A61B 1/24; A61C 7/08

(52) U.S. Cl. CPC A61C 7/002; A61B 1/24; A61C 7/08; G06F 19/3418; G06K 9/00221

(57) ABSTRACT

Provided are a method and system for providing remote teeth alignment services, and a computer-readable medium on which the method is recorded. A method of providing remote teeth alignment services using a computer system including at least one orthodontic patient terminal, an orthodontist terminal, and a server computer connected to a network includes receiving a teeth alignment report information inputted from the orthodontic patient terminal and storing the input teeth alignment report information, and transmitting the stored teeth alignment report information to the orthodontist terminal.

### BACKGROUND

### 1. Field

The present invention relates to a method and system for providing remote teeth alignment services, and a computer-readable medium on which the method is recorded and, more particularly, to a method and system for providing remote teeth alignment services to remotely advise an appropriate step of a clear aligner using a smartphone of an orthodontic patient, and a computer-readable medium on which the method is recorded.

### 2. Description of the Related Art

Orthodontic treatment may be classified into metal-brace treatment using metal braces, lingual treatment using lingual braces, and clear-aligner treatment using clear aligners.

In particular, clear-aligner treatment attracts people's attention compared to the other orthodontic treatment methods because a clear aligner covering teeth like a mouth piece is invisible, detachable, and easily wearable.

The Internet consists of a huge number of computers and computer networks for globally interconnecting the computers using communication links.

The interconnected computers exchange information using a variety of services, e.g., e-mails, Gopher, and World Wide Web (WWW). The World Wide Web allows a server computer system (e.g., a web server or a website) to transmit graphical webpages of information to a remote client computer system.

The remote client computer system may display various webpages received from the server computer.

Due to rapid development of wireless communication networks including a mobile communication network such as a second generation (2G), third generation (3G), fourth generation (4G), fifth generation (5G), or long term evolution (LTE) network, a WIFI communication network, a Bluetooth communication network, a near field communication (NFC) network, a cellular communication network, a code division multiple access (CDMA) communication network, an Ethernet communication network, a WiMAX communication network, a local area network (LAN), a wide area network (WAN), a radio frequency (RF) communication network, an infrared communication network, and an optical communication network, a variety of systems are being developed using the wireless communication networks.

Using the conventional clear-aligner treatment method, an orthodontic patient should regularly visit a dental clinic for a long period of time to allow a dentist to accurately check the state of his/her teeth and determine a currently appropriate step of a clear aligner.

However, if the dental clinic is far, if the orthodontic patient is not capable of frequently visiting the dental clinic, or if the orthodontic patient cannot visit the dental clinic any more due to going abroad to study, traveling, moving out, or the like, the orthodontic patient stops visiting the dental clinic. As such, a large number of problems occur. For example, the aligned state of teeth goes bad, and the time and cost for teeth alignment are increased. In worse cases, the orthodontic patient should continue his/her teeth alignment at another dental clinic by spending much cost and time, or can give up his/her teeth alignment.

### SUMMARY

The present invention provides a method and system for providing remote teeth alignment services, by which an orthodontist may obtain accurate teeth alignment information using a device, e.g., a smartphone, of an orthodontic patient, may remotely and accurately diagnose the condition of the patient based on the teeth alignment information, and may remotely provide information about an accurate step of a clear aligner to be currently worn by the orthodontic patient, thereby greatly reducing a diagnosis time and cost of the orthodontic patient and the orthodontist, the accuracy of diagnosis may be improved by automatically obtaining photos of accurate sizes and locations using various virtual lines, the teeth alignment information may be easily used by the orthodontist by providing the teeth alignment information in the form of report information and classifying the teeth alignment information based on time, teeth alignment may be precisely performed in a customized manner by providing manufacturing information to a clear aligner manufacturer when an additional or modified clear aligner is necessary, interest of orthodontic patients may be increased by disclosing the teeth alignment information of a selected orthodontic patient, and the orthodontic patients may be inspired with a greater desire for aligned teeth by providing benefits to the selected orthodontic patient, and a computer-readable medium having recorded the method thereon. However, the scope of the present invention is not limited thereto.

According to an aspect of the present invention, there is provided a method of providing remote teeth alignment services using a computer system including at least one orthodontic patient terminal, an orthodontist terminal, and a server computer connected to a network, the method including receiving a teeth alignment report information inputted from the orthodontic patient terminal and storing the input teeth alignment report information, and transmitting the stored teeth alignment report information to the orthodontist terminal.

According to another aspect of the present invention, there is provided a method of providing remote teeth alignment services using a computer system including at least one orthodontic patient terminal, an orthodontist terminal, and a server computer connected to a network, the server computer including a teeth alignment report information input program for receiving a teeth alignment report information inputted from the orthodontic patient terminal, a teeth alignment report information storage program for storing the input teeth alignment report information together with an input time information, a teeth alignment report information transmission program for transmitting the teeth alignment report information stored together with the input time information, to the orthodontist terminal, and a teeth alignment report information database for storing the teeth alignment report information, and the method including (a) receiving the teeth alignment report information, including photographs or videos, inputted from the orthodontic patient terminal using the teeth alignment report information input program, (b) storing the inputted teeth alignment report information together with the input time information using the teeth alignment report information storage program, and (c) transmitting the teeth alignment report information stored together with the input time information, to the orthodontist terminal using the teeth alignment report information transmission program.

The teeth alignment report information may include at least one of photographing step information, photographing date information, and a front-view teeth photo, a bottom-view low-angle teeth photo, a front-view automatically-recognized mouth gag photo, a front-view automatically-recognized face contour photo, a left-side-angle automatically-recognized mouth gag photo, a left-side-angle automatically-recognized face contour photo, a right-side-angle automatically-recognized mouth gag photo, a right-side-angle automatically-recognized face contour photo, a collection of top, bottom, left, and right side photos surrounding a central front photo, a low-angle upper jaw photo, a high-angle lower jaw photo, and combinations thereof, which are taken using a camera of the orthodontic patient terminal.

The teeth alignment report information input program may include at least one of a camera activation program for activating a camera of the orthodontic patient terminal, a current step information input program, a current date information input program, a text guidance information display program for displaying text guidance information to an orthodontic patient, a voice guidance information output program for outputting voice guidance information to the orthodontic patient, a face contour automatic-recognition photographing program for displaying a virtual face contour line on a screen of the orthodontic patient terminal and automatically recognizing and taking a photo of a face contour of the orthodontic patient shown on the screen, a teeth contour automatic-recognition photographing program for displaying a virtual teeth line on the screen of the orthodontic patient terminal and automatically recognizing and taking a photo of teeth of the orthodontic patient shown on the screen, a mouth gag contour automatic-recognition photographing program for displaying a virtual mouth gag contour line on the screen of the orthodontic patient terminal and automatically recognizing and taking a photo of a mouth gag shown on the screen, a photo check program for displaying the taken photos on the screen, and combinations thereof.

The server computer may further include a teeth alignment guidance information transmission program for receiving a teeth alignment guidance information from the orthodontist terminal and transmitting the teeth alignment guidance information to the orthodontic patient terminal, and a teeth alignment guidance information database for storing the teeth alignment guidance information, and the method may further include (d) receiving the teeth alignment guidance information from the orthodontist terminal and transmitting the teeth alignment guidance information to the orthodontic patient terminal using the teeth alignment guidance information transmission program, after step (c).

The teeth alignment guidance information may include at least one of a clear aligner information about a clear aligner to be worn in a current step among a clear aligner set, a wearing time information about a wearing time of the clear aligner, a replacement information about replacement of a part of or the whole clear aligner set, a clear aligner state information about the state of the clear aligner, a clear aligner usage guidance information, a question information, a memo information, a clear aligner precautions information, a dental clinic visit information, and combinations thereof.

The server computer may further include a smile information transmission program for generating a virtual smile information of an orthodontic patient based on the teeth alignment report information and transmitting the virtual smile information to the orthodontic patient terminal, and a smile information database for storing the virtual smile information, and the method may further include (e) generating the virtual smile information of the orthodontic patient based on the teeth alignment report information and transmitting the virtual smile information to the orthodontic patient terminal or the orthodontist terminal using the smile information transmission program, after step (b).

The server computer may further include a clear aligner information transmission program for receiving a clear aligner information about a currently worn clear aligner from the orthodontic patient terminal, storing the clear aligner information, and transmitting the clear aligner information to the orthodontist terminal, and a clear aligner information database for storing the clear aligner information, and the method may further include (f) receiving the clear aligner information about a currently worn clear aligner from the orthodontic patient terminal and transmitting the clear aligner information to the orthodontist terminal using the clear aligner information transmission program, after step (b).

The teeth alignment report information transmission program may include at least one of a patient-specific information transmission program for transmitting a teeth alignment information per orthodontic patient, a current step information transmission program for transmitting a current clear aligner step information, a photographing schedule transmission program for transmitting a photographing schedule information, a schedule adjustment information transmission program for adjusting the photographing schedule of the orthodontic patient, a calendar information transmission program for transmitting a calendar information per orthodontic patient, an individual memo transmission program for transmitting a memo information per orthodontic patient, an integrated memo information transmission program for transmitting a memo information to all orthodontic patients, and combinations thereof.

The server computer may further include a clear aligner manufacturing program for transmitting a clear aligner manufacturing information to a clear aligner manufacturer terminal upon determining that an additional or modified clear aligner is needed, based on the teeth alignment report information, and a clear aligner manufacturing information database for storing the clear aligner manufacturing information, and the method may further include (g) transmitting the clear aligner manufacturing information to the clear aligner manufacturer terminal upon determining that an additional or modified clear aligner is needed, based on the teeth alignment report information using the clear aligner manufacturing program, after step (c).

The server computer may further include an information disclosure program for transmitting the teeth alignment report information, disclosure of which is permitted by an orthodontic patient, to other orthodontic patient terminals, and the method may further include (h) transmitting the teeth alignment report information, disclosure of which is permitted by the orthodontic patient, to the other orthodontic patient terminals using the information disclosure program, after step (c).

The server computer may further include an evaluation program for generating an evaluation result information about the teeth alignment report information, selecting an orthodontic patient having a good evaluation result based on the evaluation result information, and transmitting a reward information to the orthodontic patient terminal of the selected orthodontic patient, and an evaluation result information database for storing the evaluation result information, and the method may further include (i) generating the evaluation result information about the teeth alignment report information, selecting an orthodontic patient having a good evaluation result based on the evaluation result information, and transmitting the reward information to the orthodontic patient terminal of the selected orthodontic patient using the evaluation program, after step (c).

According to another aspect of the present invention, there is provided a computer system for providing remote teeth alignment services using at least one orthodontic patient terminal, an orthodontist terminal, and a server computer connected to a network, the server computer including a teeth alignment report information input program for receiving a teeth alignment report information inputted from the orthodontic patient terminal, a teeth alignment report information storage program for storing the input teeth alignment report information together with an input time information, a teeth alignment report information transmission program for transmitting the teeth alignment report information stored together with the input time information, to the orthodontist terminal, a teeth alignment report information database for storing the teeth alignment report information, and a controller programmed to receive the teeth alignment report information, including photographs or videos, inputted from the orthodontic patient terminal using the teeth alignment report information input program, to store the input teeth alignment report information together with the input time information using the teeth alignment report information storage program, and to transmit the teeth alignment report information stored together with the input time information, to the orthodontist terminal using the teeth alignment report information transmission program.

According to another aspect of the present invention, there is provided a computer-readable medium on which is recorded a method of providing remote teeth alignment services using a computer system including at least one orthodontic patient terminal, an orthodontist terminal, and a server computer connected to a network, the server computer including a teeth alignment report information input program for receiving a teeth alignment report information inputted from the orthodontic patient terminal, a teeth alignment report information storage program for storing the input teeth alignment report information together with an input time information, a teeth alignment report information transmission program for transmitting the teeth alignment report information stored together with the input time information, to the orthodontist terminal, and a teeth alignment report information database for storing the teeth alignment report information, and the method including (a) receiving the teeth alignment report information, including photographs or videos, inputted from the orthodontic patient terminal using the teeth alignment report information input program, (b) storing the inputted teeth alignment report information together with the input time information using the teeth alignment report information storage program, and (c) transmitting the teeth alignment report information stored together with the input time information, to the orthodontist terminal using the teeth alignment report information transmission program.

### BRIEF DESCRIPTION OF THE DRAWINGS

The above and other features and advantages of the present invention will become more apparent by describing in detail embodiments thereof with reference to the attached drawings in which:

FIG. 1 is a conceptual view of a system for providing remote teeth alignment services, according to some embodiments of the present invention;

FIG. 2 is a detailed block diagram of a teeth alignment report information input program of the remote teeth alignment service providing system of FIG. 1;

FIG. 3 is a detailed block diagram of a teeth alignment report information transmission program of the remote teeth alignment service providing system of FIG.

FIG. 4 is a conceptual view showing the relationship among an operator for providing remote teeth alignment services of the present invention, an orthodontic patient, an orthodontist, and a clear aligner manufacturer;

FIG. 5 is a flowchart of a method of providing remote teeth alignment services, according to some embodiments of the present invention;

FIG. 6 is a flowchart of a method of providing remote teeth alignment services, according to other embodiments of the present invention; and

FIGS. 7 to 23 are examples of images displayed on screens of devices using the remote teeth alignment service providing system according to some embodiments of the present invention.

### DETAILED DESCRIPTION

Hereinafter, the present invention will be described in detail by explaining embodiments of the invention with reference to the attached drawings.

The invention may, however, be embodied in many different forms and should not be construed as being limited to the embodiments set forth herein; rather, these embodiments are provided so that this disclosure will be thorough and complete, and will fully convey the concept of the invention to one of ordinary skill in the art. In the drawings, the thicknesses or sizes of layers are exaggerated for clarity.

The terminology used herein is for the purpose of describing particular embodiments only and is not intended to be limiting of embodiments. As used herein, the singular forms “a,” “an” and “the” are intended to include the plural forms as well, unless the context clearly indicates otherwise. It will be further understood that the terms “comprises” and/or “comprising” when used in this specification, specify the presence of stated features, integers, steps, operations, elements, and/or components, but do not preclude the presence or addition of one or more other features, integers, steps, operations, elements, components, and/or groups thereof.

Embodiments of the invention are described herein with reference to schematic illustrations of idealized embodiments (and intermediate structures) of the invention. As such, variations from the shapes of the illustrations as a result, for example, of manufacturing techniques and/or tolerances, are to be expected. Thus, the embodiments of the invention should not be construed as limited to the particular shapes of regions illustrated herein, but are to include deviations in shapes that result, for example, from manufacturing.

A method and system for providing remote teeth alignment services, and a computer-readable medium having recorded the method thereon, according to some embodiments of the present invention are now described in detail with reference to the attached drawings.

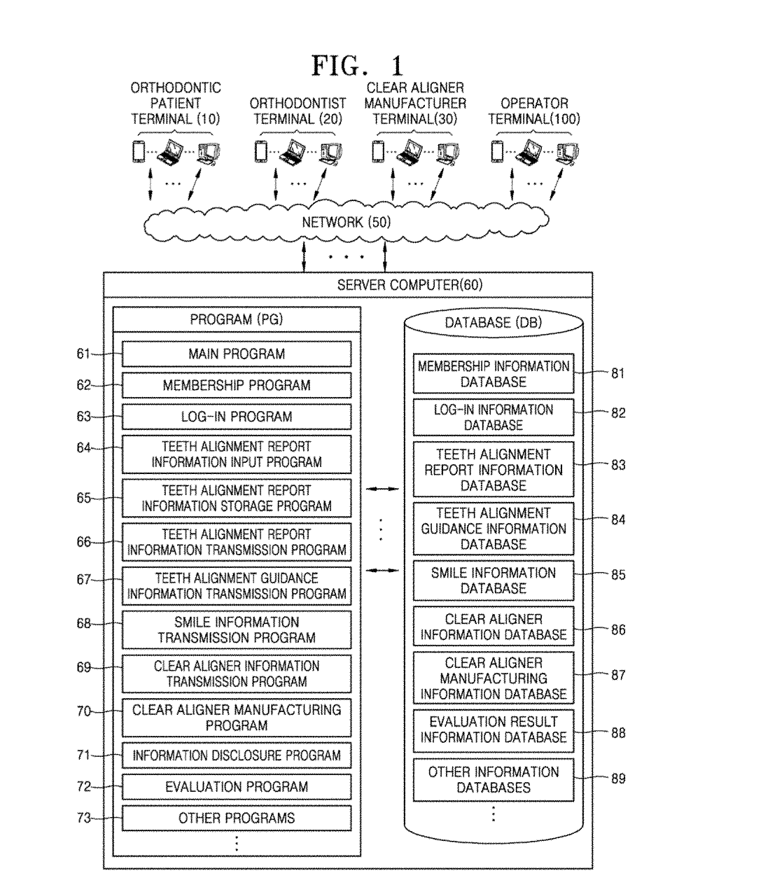

FIG. 1 is a conceptual view of a system for providing remote teeth alignment services, according to some embodiments of the present invention.

Initially, as illustrated in FIG. 1, the remote teeth alignment service providing system according to some embodiments of the present invention mainly includes an orthodontic patient terminal 10, an orthodontist terminal 20, a clear aligner manufacturer terminal 30, an operator terminal 100, and a server computer 60 connected to a network 50.

Herein, the server computer 60 is programmed to receive a teeth alignment report information inputted from the orthodontic patient terminal 10, to store the input teeth alignment report information, and to transmit the stored teeth alignment report information to the orthodontist terminal 20, and may be a computer provided in a company, an agency, or a franchise capable of providing remote teeth alignment services through the network 50.

For example, the orthodontic patient terminal 10 may be a device of an orthodontic patient who desires to remotely receive information about an accurate step of a clear aligner to be currently worn by the orthodontic patient, using the system of the present invention.

Herein, the orthodontic patient terminal 10 is not limited to a smartphone and may be one of all information devices capable of transmitting a teeth alignment information. For example, the orthodontic patient terminal 10 may be a personal computer, a mobile device, a personal digital assistant (PDA), a smart pad, a laptop computer, a smart camera, a smart camcorder, an electronic book, a smart scanner, or the like.

For example, the orthodontist terminal 20 may be a device of an orthodontist, e.g., a dentist, a dental nurse, or a dental professional, who desires to remotely provide information about an accurate step of a clear aligner to be worn by the orthodontic patient, to the orthodontic patient using the system of the present invention.

Herein, the orthodontist terminal 20 is not limited to a smartphone and may be one of all information devices capable of transmitting a teeth alignment guidance information, e.g., information about an accurate step of a clear aligner to be worn by the orthodontic patient, to the orthodontic patient. For example, the orthodontist terminal 20 may be a personal computer, a mobile device, a PDA, a smart pad, a laptop computer, a smart camera, a smart camcorder, an electronic book, a smart scanner, or the like.

For example, the clear aligner manufacturer terminal 30 may be a computer or a smartphone provided in a company, an agency, or a franchise which desires to manufacture a clear aligner set and added or modified clear aligners to be provided to the orthodontic patient, using the system of the present invention.

Herein, the clear aligner manufacturer terminal 30 is not limited to a smartphone and may be one of all information devices capable of receiving information about manufacturing of clear aligners. For example, the clear aligner manufacturer terminal 30 may be a personal computer, a mobile device, a PDA, a smart pad, a laptop computer, a smart camera, a smart camcorder, an electronic book, a smart scanner, or the like.

For example, the operator terminal 100 is a device of a person, a company, an agency, or a franchise for managing and operating the server computer 60, is not limited to a computer or a smartphone, and may be an information device, a PDA, a smartphone, a smart pad, a camera, a camcorder, a laptop computer, an electronic book, a personal computer, or another server computer capable of receiving various types of text information, number information, or image information, and of selecting various commands.

Although the clear aligner manufacturer terminal 30 and the operator terminal 100 are separately managed in FIG. 1, the present invention is not limited thereto. For example, the above-described operator terminal 100 may also serve as the clear aligner manufacturer terminal 30. That is, the operator may also be the clear aligner manufacturer. In addition, the operator may also be the orthodontist.

The orthodontic patient terminal 10, the orthodontist terminal 20, the clear aligner manufacturer terminal 30, the operator terminal 100, and the server computer 60 of FIG. 1 are interconnected through the network 50 using various applications, hybrid applications, or programs installed thereon. The devices 10, 20, 30, and 40 interconnected through the network 50 may use a conventional mobile communication network such as a second generation (2G), third generation (3G), or fourth generation (4G) network, a WIFI communication network, a Bluetooth communication network, a near field communication (NFC) network, a cellular communication network, a code division multiple access (CDMA) communication network, a long term evolution (LTE) communication network, an Ethernet communication network, a WiMAX communication network, a local area network (LAN), a wide area network (WAN), a radio frequency (RF) communication network, an infrared communication network, or an optical communication network, and may have an Internet browser (e.g., Netscape or Internet Explorer) capable of displaying websites in the form of HTML, XML, or HTML5, an internal, external, or short-distance/long-distance wired/wireless network access protocol device, or the like.

As illustrated in FIG. 1, the server computer 60 includes a program controller PG for controlling programs, and a database DB for storing various types of information.

Particularly, as illustrated in FIG. 1, the program controller PG may include a main program 61 for managing all other programs, a membership program 62 for receiving a membership information from the orthodontic patient terminal 10 or the orthodontist terminal 20 and storing the membership information, a log-in program 63 for receiving a log-in information from the orthodontic patient terminal 10 or the orthodontist terminal 20, a teeth alignment report information input program 64 for receiving a teeth alignment report information inputted from the orthodontic patient terminal 10, a teeth alignment report information storage program 65 for storing the input teeth alignment report information together with an input time information, a teeth alignment report information transmission program 66 for transmitting the teeth alignment report information stored together with the input time information, to the orthodontist terminal 20, a teeth alignment guidance information transmission program 67 for receiving a teeth alignment guidance information from the orthodontist terminal 20 and transmitting the teeth alignment guidance information to the orthodontic patient terminal 10, a smile information transmission program 68 for generating a virtual smile information of an orthodontic patient based on the teeth alignment report information and transmitting the virtual smile information to the orthodontic patient terminal 10, a clear aligner information transmission program 69 for receiving a clear aligner information about a currently worn clear aligner from the orthodontic patient terminal 10, storing the clear aligner information, and transmitting the clear aligner information to the orthodontist terminal 20, a clear aligner manufacturing program 70 for transmitting a clear aligner manufacturing information to the clear aligner manufacturer terminal 30 upon determining that an additional or modified clear aligner is needed, based on the teeth alignment report information, an information disclosure program 71 for transmitting the teeth alignment report information, disclosure of which is permitted by the orthodontic patient, to other orthodontic patient terminals 10, an evaluation program 72 for generating an evaluation result information about the teeth alignment report information, selecting an orthodontic patient having a good evaluation result based on the evaluation result information, and transmitting a reward information to the orthodontic patient terminal 10 of the selected orthodontic patient, and other programs 73 for performing other functions, e.g., home page management, promotion, advertisement, data processing, cost management, and application installation.

Herein, the main program 61 manages all other programs. The main program 61 may be implemented in the form of an on-line home page or a main page of an application, and may be a program for controlling all other programs by receiving various command signals from the orthodontic patient terminal 10, the orthodontist terminal 20, the clear aligner manufacturer terminal 30, and the operator terminal 100.

The membership program 62 may be a program for registering the orthodontic patient, the orthodontist, and the clear aligner manufacturer as members. The membership program 62 may provide standard terms or terms for collecting and using personal information and may get an agreement thereof to receive the membership information from the orthodontic patient terminal 10 or the orthodontist terminal 20 and to store the membership information. Herein, the membership information may include at least one of a real name, an Internet personal identification number (i-PIN), an identifier (ID), a password, an e-mail, a mobile phone number, an address, personal information, a department name, a position name, an employee identification number, a resident registration number, and a serial number.

The log-in program 63 may be a program for receiving the log-in information, e.g., an ID or a password, required when the orthodontic patient, the orthodontist, or the clear aligner manufacturer logs in the server computer 60, from the orthodontic patient terminal 10 or the orthodontist terminal 20.

The teeth alignment report information input program 64 may be a program for receiving the teeth alignment report information acquired using a camera of the orthodontic patient terminal 10 and showing a teeth alignment state of the orthodontic patient.

Herein, for example, the teeth alignment report information may include at least one of photographing step information, photographing date information, and a front-view teeth photo, a bottom-view low-angle teeth photo, a front-view automatically-recognized mouth gag photo, a front-view automatically-recognized face contour photo, a left-side-angle automatically-recognized mouth gag photo, a left-side-angle automatically-recognized face contour photo, a right-side-angle automatically-recognized mouth gag photo, a right-side-angle automatically-recognized face contour photo, a collection of top, bottom, left, and right side photos surrounding a central front photo, a low-angle upper jaw photo, a high-angle lower jaw photo, and combinations thereof, which are taken using the camera of the orthodontic patient terminal 10 to show the teeth alignment state of the orthodontic patient.

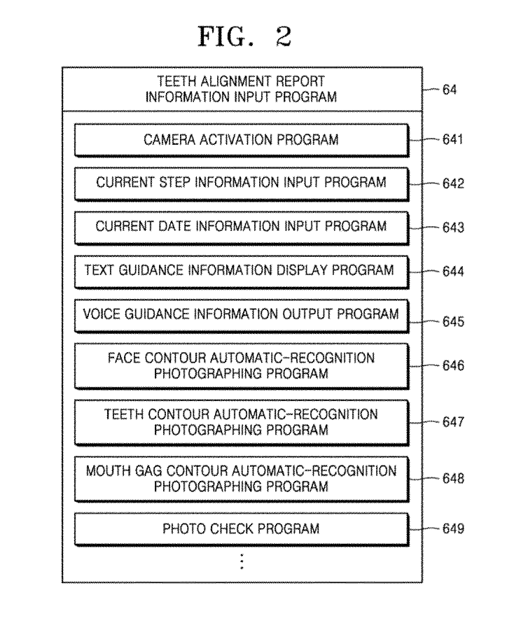

FIG. 2 is a detailed block diagram of the teeth alignment report information input program 64 of the remote teeth alignment service providing system of FIG. 1.

Specifically, for example, as illustrated in FIG. 2, the teeth alignment report information input program 64 may include at least one of a camera activation program 641 for activating the camera of the orthodontic patient terminal 10, a current step information input program 642, a current date information input program 643, a text guidance information display program 644 for displaying a text guidance information to the orthodontic patient, a voice guidance information output program 645 for outputting a voice guidance information to the orthodontic patient, a face contour automatic-recognition photographing program 646 for displaying a virtual face contour line on a screen of the orthodontic patient terminal 10 and automatically recognizing and taking a photo of a face contour of the orthodontic patient shown on the screen, a teeth contour automatic-recognition photographing program 647 for displaying a virtual teeth line on the screen of the orthodontic patient terminal 10 and automatically recognizing and taking a photo of teeth of the orthodontic patient shown on the screen, a mouth gag contour automatic-recognition photographing program 648 for displaying a virtual mouth gag contour line on the screen of the orthodontic patient terminal 10 and automatically recognizing and taking a photo of a mouth gag shown on the screen, a photo check program 649 for displaying the taken photos on the screen, and combinations thereof.

The teeth alignment report information storage program 65 may be a program for storing the input teeth alignment report information together with the input time information, and the teeth alignment report information may be classified and stored per orthodontic patient, per date, per orthodontist in charge, per calendar, per previous data, per previous memo, per previous message, etc.

The teeth alignment report information transmission program 66 may be a program for transmitting the teeth alignment report information stored together with the input time information, to the orthodontist terminal 20 in a wireless or wired manner for remote teeth alignment.

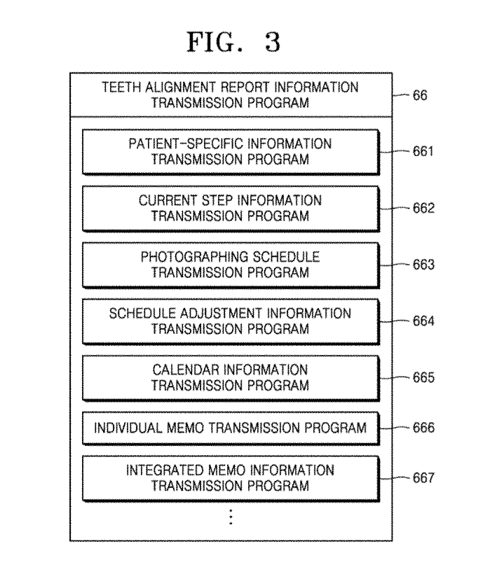

FIG. 3 is a detailed block diagram of the teeth alignment report information transmission program 66 of the remote teeth alignment service providing system of FIG. 1.

Specifically, for example, as illustrated in FIG. 3, the teeth alignment report information transmission program 66 may include at least one of a patient-specific information transmission program 661 for transmitting a teeth alignment information per orthodontic patient, a current step information transmission program 662 for transmitting a current clear aligner step information, a photographing schedule transmission program 663 for transmitting a photographing schedule information, a schedule adjustment information transmission program 664 for adjusting the photographing schedule of the orthodontic patient, a calendar information transmission program 665 for transmitting a calendar information per orthodontic patient, an individual memo transmission program 666 for transmitting a memo information per orthodontic patient, an integrated memo information transmission program 667 for transmitting a memo information to all orthodontic patients, and combinations thereof.

The teeth alignment guidance information transmission program 67 may be a program for receiving the teeth alignment report information and then transmitting the teeth alignment guidance information received from the orthodontist terminal 20, to the orthodontic patient terminal 10 in such a manner that the orthodontist accurately determines a current teeth alignment state of the orthodontist and advises, for example, an appropriate step of a clear aligner to be currently worn by the orthodontic patient.

Herein, for example, the teeth alignment guidance information may include at least one of a clear aligner information about a clear aligner to be worn in a current step among a clear aligner set, a wearing time information about a wearing time of the clear aligner, a replacement information about replacement of a part of or the whole clear aligner set, a clear aligner state information about the state of the clear aligner, a clear aligner usage guidance information, a question information, a memo information, a clear aligner precautions information, a dental clinic visit information, and combinations thereof.

The smile information transmission program 68 may be a program for generating the virtual smile information, i.e., a smiling face photo or video, before and after teeth alignment of the orthodontic patient based on the teeth alignment report information, and transmitting the virtual smile information to the orthodontic patient terminal 10 as an additional service. Accordingly, the orthodontic patient may previously check a virtual face, i.e., a smile face, after teeth alignment and thus may be inspired with a greater desire for aligned teeth.

The clear aligner information transmission program 69 may be a program for receiving the clear aligner information about a currently worn clear aligner from the orthodontic patient terminal 10, storing the clear aligner information, and transmitting the clear aligner information to the orthodontist terminal 20 in such a manner that the orthodontist checks a photo or a video of the clear aligner currently worn by the orthodontic patient, and determines whether the clear aligner is normal and corresponds to an appropriate step.

The clear aligner manufacturing program 70 may be a program for transmitting the clear aligner manufacturing information to the clear aligner manufacturer terminal 30 upon determining that an additional or modified clear aligner is needed, based on the teeth alignment report information, in such a manner that the clear aligner manufacturer manufactures an additional or modified clear aligner.

The information disclosure program 71 may be a program for transmitting the teeth alignment report information, disclosure of which is permitted by the orthodontic patient, to other orthodontic patient terminals 10, in such a manner that the teeth alignment report information of a celebrity, e.g., a singer, a movie star, an actor, or a comedian, or a person having straight teeth is disclosed by permission. The orthodontic patient who permits to disclose the teeth alignment report information thereof may receive a teeth alignment service at a discount or at no cost.

The evaluation program 72 may be a program for generating the evaluation result information about the teeth alignment report information through a vote, a user preference survey, a rating, an opinion poll, or the like of the orthodontist or the orthodontic patients, selecting an orthodontic patient having a good evaluation result as an excellent patient, a diligent patient, an honored patient, or the like based on the evaluation result information, and transmitting the reward information to the orthodontic patient terminal 10 of the selected orthodontic patient to reward the orthodontic patient with, for example, e-money, a product, money, points, a coupon, or a ticket.

Herein, the above-described programs may be executive programs downloaded or installed in the orthodontic patient terminal 10, the orthodontist terminal 20, the clear aligner manufacturer terminal 30, or the operator terminal 100, and may operate in association with user applications.

However, the above-described programs are not limited to the executive programs or association with smartphone applications, and may operate in association with various types of devices.

Accordingly, an orthodontist, e.g., a dentist, may obtain accurate teeth alignment information using a device, e.g., a smartphone, of a patient, i.e., an orthodontic patient, may remotely and accurately diagnose the condition of the patient based on the teeth alignment information, and may remotely provide information about an accurate step of a clear aligner to be currently worn by the orthodontic patient, thereby greatly reducing a diagnosis time and cost of the orthodontic patient and the orthodontist. The accuracy of diagnosis may be improved by automatically obtaining photos of accurate sizes and locations using various identifiers such as virtual lines. The teeth alignment information may be easily used by the orthodontist by providing the teeth alignment information in the form of report information and classifying the teeth alignment information based on time. Teeth alignment may be precisely performed in a customized manner by providing manufacturing information to a clear aligner manufacturer when an additional or modified clear aligner is necessary. Interest of orthodontic patients may be increased by disclosing the teeth alignment information of a celebrity or a selected orthodontic patient, and the orthodontic patients may be inspired with a greater desire for aligned teeth by providing benefits to an orthodontic patient selected as an excellent patient or the like.

As illustrated in FIG. 1, the database DB may include a membership information database 81 for storing the membership information, a log-in information database 82 for storing the log-in information, a teeth alignment report information database 83 for storing the teeth alignment report information, a teeth alignment guidance information database 84 for storing the teeth alignment guidance information, a smile information database 85 for storing the virtual smile information, a clear aligner information database 86 for storing the clear aligner information, a clear aligner manufacturing information database 87 for storing the clear aligner manufacturing information, an evaluation result information database 88 for storing the evaluation result information, and other information databases 89 for storing other information.

Accordingly, the server computer 60 may provide remote teeth alignment services for receiving the teeth alignment report information, including photographs or videos, inputted from the orthodontic patient terminal 10 using the teeth alignment report information input program 64, storing the inputted teeth alignment report information together with the input time information using the teeth alignment report information storage program 65, generating the virtual smile information of the orthodontic patient based on the teeth alignment report information and transmitting the virtual smile information to the orthodontic patient terminal 10 or the orthodontist terminal 20 using the smile information transmission program 68, receiving the clear aligner information about a currently worn clear aligner from the orthodontic patient terminal 10 and transmitting the clear aligner information to the orthodontist terminal 20 using the clear aligner information transmission program 69, transmitting the teeth alignment report information stored together with the input time information, to the orthodontist terminal 20 using the teeth alignment report information transmission program 66, receiving the teeth alignment guidance information from the orthodontist terminal 20 and transmitting the teeth alignment guidance information to the orthodontic patient terminal 10 using the teeth alignment guidance information transmission program 67, transmitting the clear aligner manufacturing information to the clear aligner manufacturer terminal 30 upon determining that that additional or modified clear aligner is needed, based on the teeth alignment report information using the clear aligner manufacturing program 70, transmitting the teeth alignment report information, disclosure of which is permitted by the orthodontic patient, to the other orthodontic patient terminals 10 using the information disclosure program 71, and generating the evaluation result information about the teeth alignment report information, selecting an orthodontic patient having a good evaluation result based on the evaluation result information, and transmitting the reward information to the orthodontic patient terminal 10 of the selected orthodontic patient using the evaluation program 72.

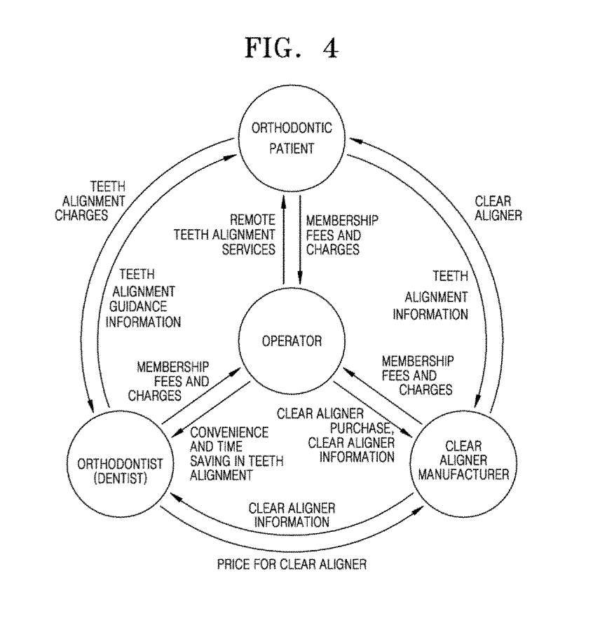

FIG. 4 is a conceptual view showing the relationship among the operator for providing remote teeth alignment services of the present invention, the orthodontic patient, the orthodontist, and the clear aligner manufacturer.

Therefore, as illustrated in FIG. 4, the operator for operating the server computer 60 may receive membership fees and charges from the orthodontic patient, the orthodontist, and the clear aligner manufacturer to provide remote teeth alignment services. The orthodontic patient, the orthodontist, and the clear aligner manufacturer may receive high-quality remote teeth alignment services, may greatly reduce times and costs required for teeth alignment, diagnosis, and clear aligner manufacturing, and clear aligner prices to be paid, and may easily receive additional or modified clear aligners and various types of information and convenience. Therefore, a business model which is advantageous and useful to all may be provided.

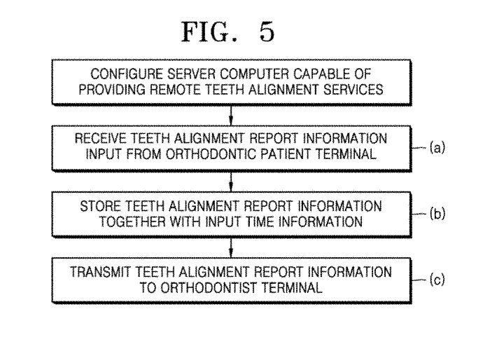

FIG. 5 is a flowchart of a method of providing remote teeth alignment services, according to some embodiments of the present invention.

As illustrated in FIGS. 1 to 5, the remote teeth alignment service providing method according to some embodiments of the present invention sequentially includes configuring the server computer 60 capable of providing remote teeth alignment services, (a) receiving the teeth alignment report information, including photographs or videos, inputted from the orthodontic patient terminal 10 using the teeth alignment report information input program 64, (b) storing the input teeth alignment report information together with the input time information using the teeth alignment report information storage program 65, and (c) transmitting the teeth alignment report information stored together with the input time information, to the orthodontist terminal 20 using the teeth alignment report information transmission program 66.

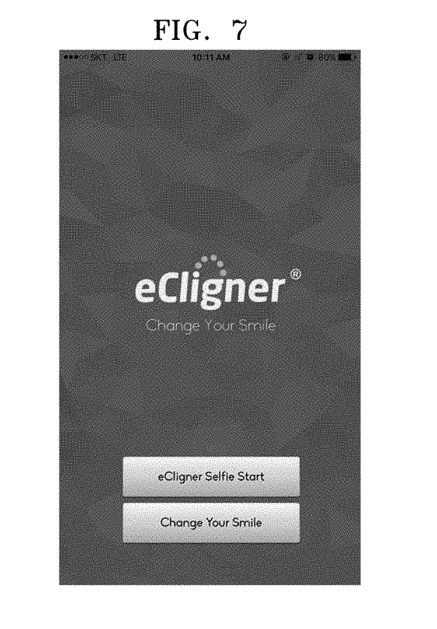

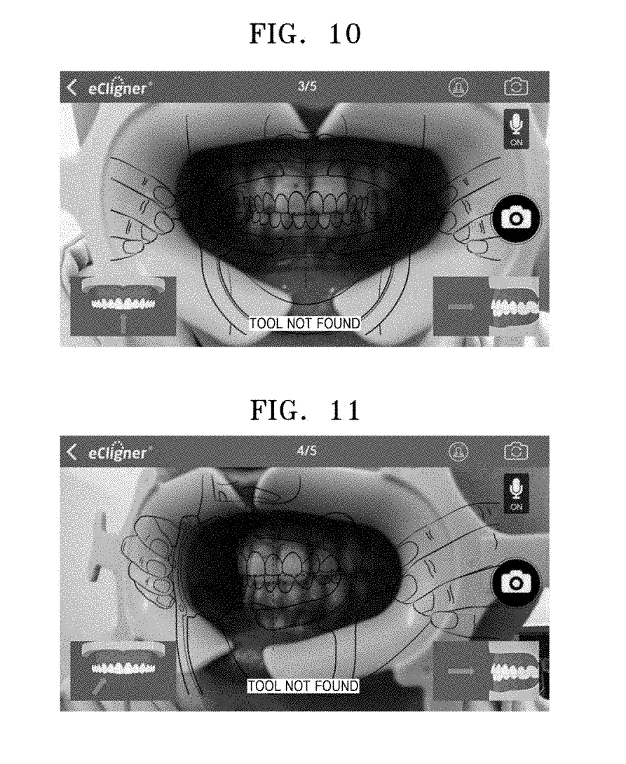

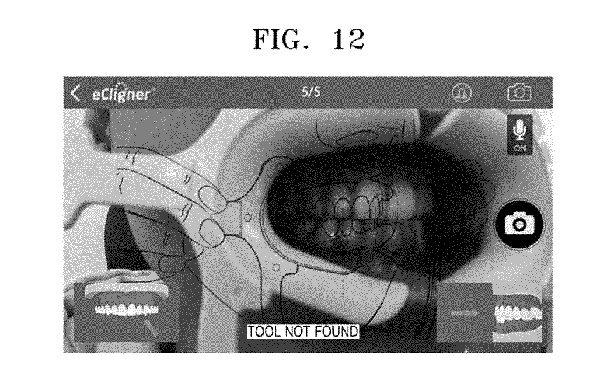

FIGS. 7 to 23 are examples of images displayed on screens of devices using the remote teeth alignment service providing system according to some embodiments of the present invention.

Specifically, for example, in (a) the receiving of the teeth alignment report information, including photographs or videos, inputted from the orthodontic patient terminal 10, initially, an orthodontic patient who is registered as a member using a main page or a smartphone application may activate and log into the main program 61 as illustrated in FIG. 7.

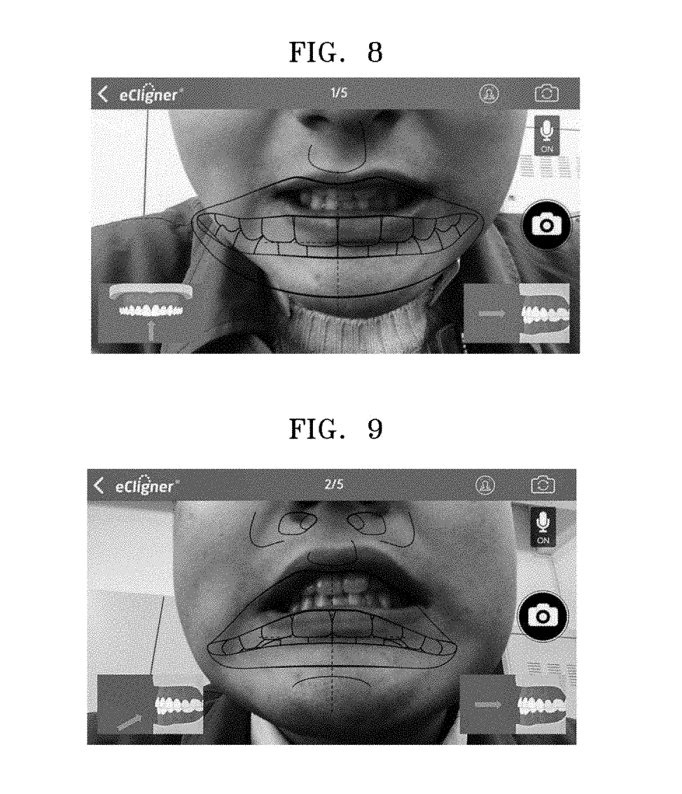

Then, as illustrated in FIGS. 8 to 12, the orthodontic patient may automatically take a photo of his/her teeth, which are being aligned, using a selfie function of a smartphone.

In this case, text guidance information may be displayed on the orthodontic patient terminal 10 using the above-described text guidance information display program 644, or voice guidance information may be outputted to orthodontic patient using the voice guidance information output program 645, and virtual face contour lines, virtual teeth lines, virtual mouth gag contour lines, etc. may be displayed as illustrated in FIGS. 8 to 12 and images displayed on the screen may be automatically-recognized and captured.

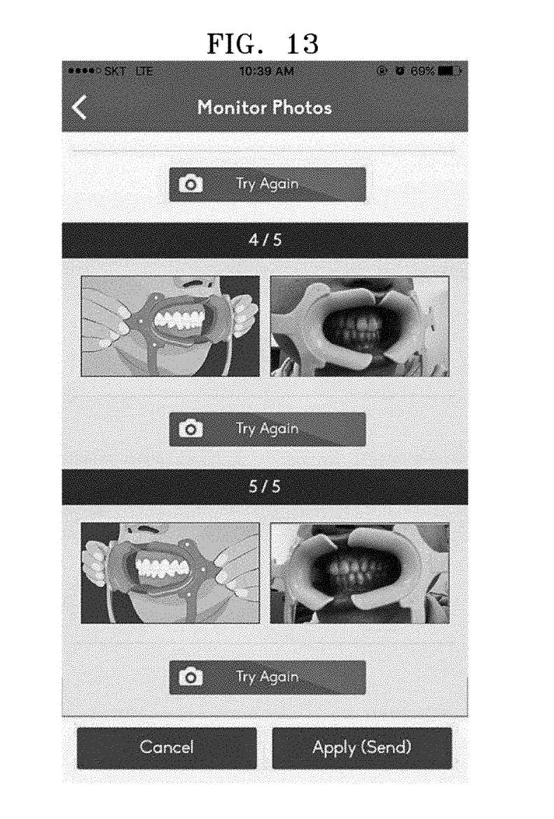

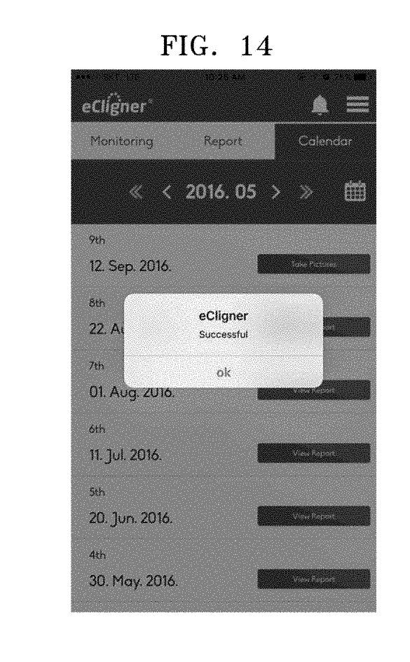

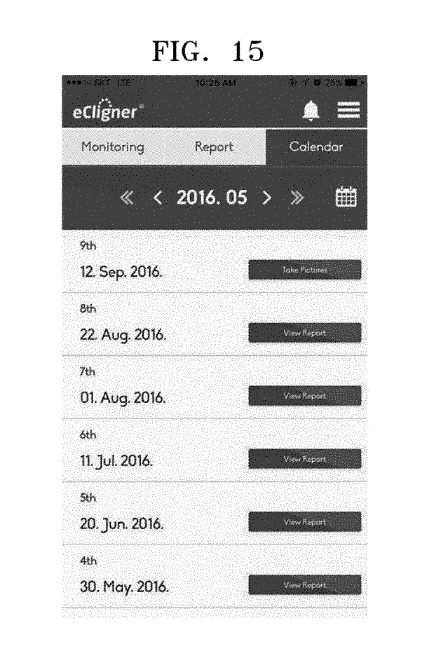

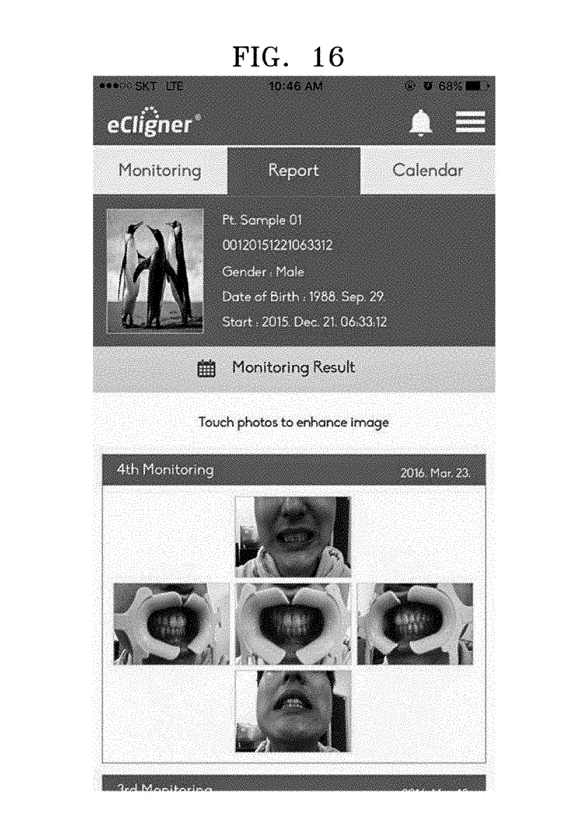

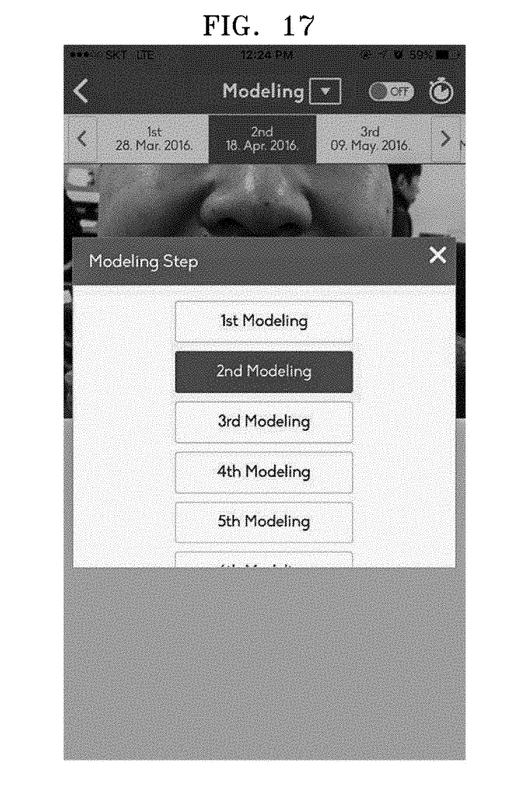

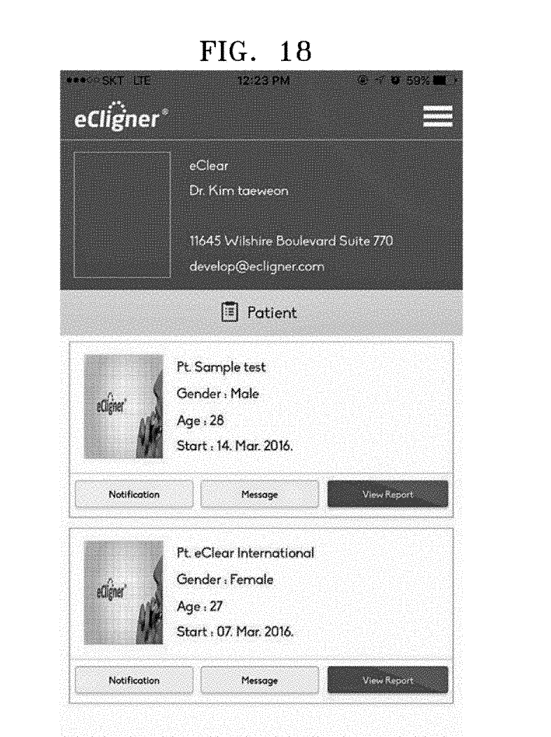

Then, in (b) the storing of the input teeth alignment report information together with the input time information, the orthodontic patient having checked the taken photos as illustrated in FIG. 13 may transmit the photos as illustrated in FIG. 14. In addition, the orthodontic patient may receive a large variety of services. For example, the orthodontic patient may view teeth alignment results per date as illustrated in FIG. 15, in the form of a report as illustrated in FIG. 16, or in the form of automatic slides as illustrated in FIG. 17. Besides, the orthodontic patient may check various notifications, ask various questions, check log-in information of other people, and check exchanged messages.

Then, in (c) the transmitting of the teeth alignment report information stored together with the input time information, to the orthodontist terminal 20, an orthodontist, e.g., a dentist, who is registered as a member using a main page or a smartphone application may activate and log in the main program 61 as illustrated in FIG. 18.

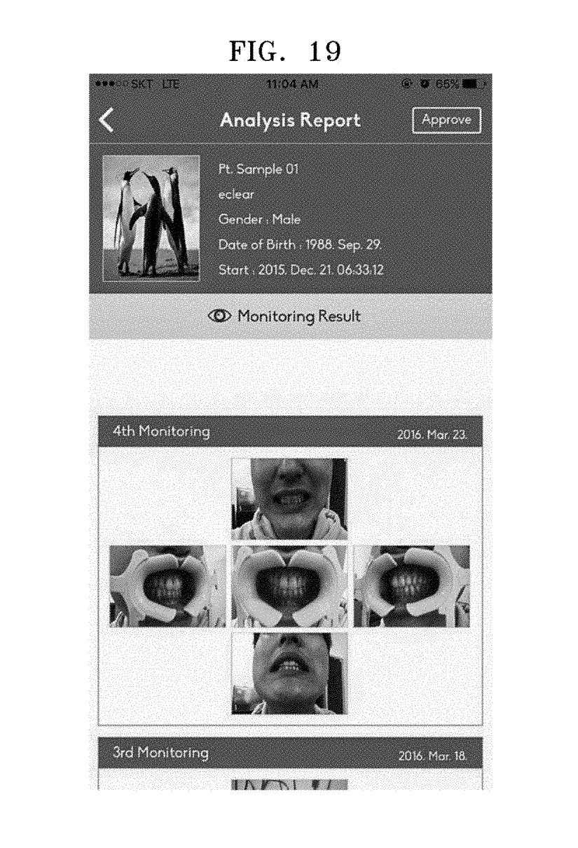

Then, as illustrated in FIG. 19, the orthodontist may check the transmitted teeth alignment report information per patient using the orthodontist terminal 20.

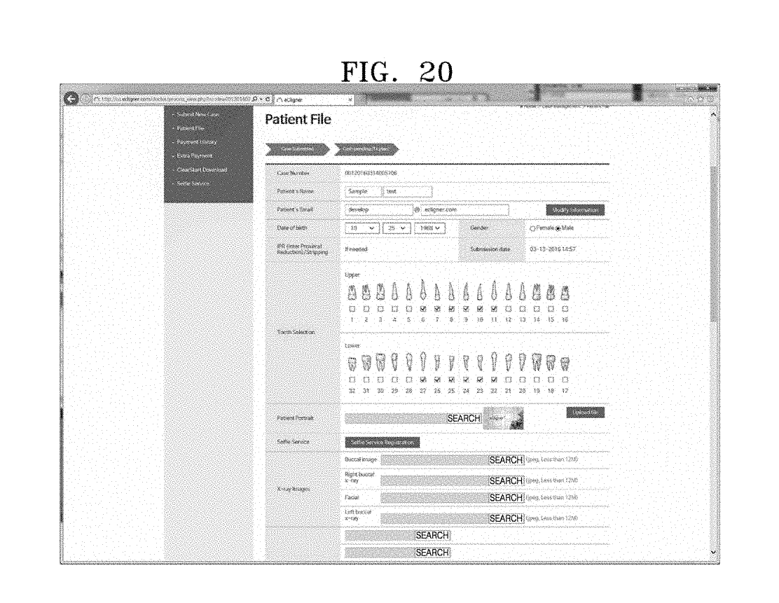

Herein, the orthodontist may check the teeth alignment report information using not only an application of a smartphone but also a home page or a program implemented in a personal computer as illustrated in FIG. 20.

Accordingly, the orthodontist may remotely provide accurate diagnosis by checking a front-view teeth photo, a bottom-view low-angle teeth photo, a front-view automatically-recognized mouth gag photo, a front-view automatically-recognized face contour photo, a left-side-angle automatically-recognized mouth gag photo, a left-side-angle automatically-recognized face contour photo, a right-side-angle automatically-recognized mouth gag photo, a right-side-angle automatically-recognized face contour photo, a collection of top, bottom, left, and right side photos surrounding a central front photo, a low-angle upper jaw photo, a high-angle lower jaw photo, etc. capable of showing the teeth alignment state of the orthodontic patient.

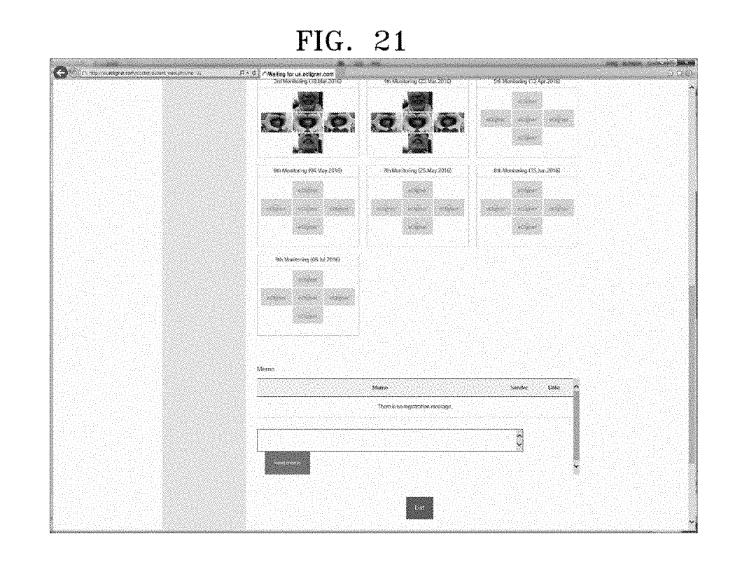

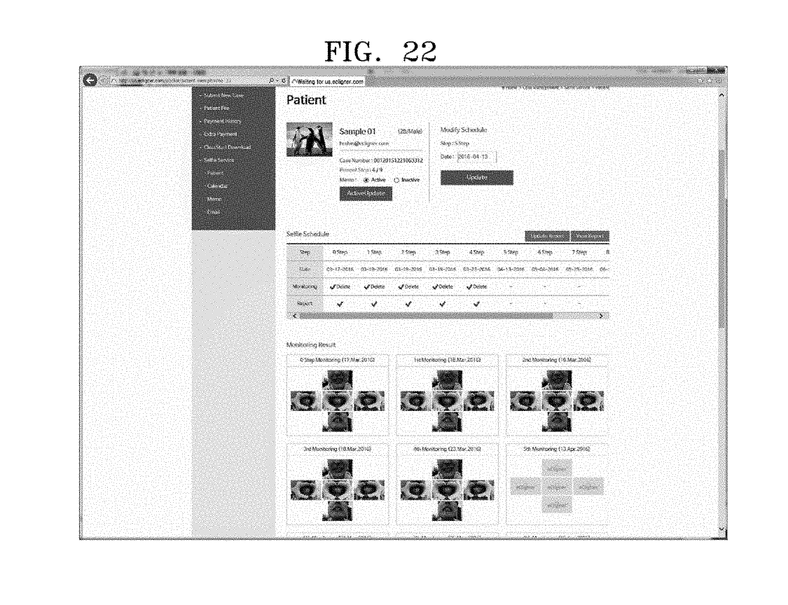

In addition, the orthodontist may schedule services to monitor a plurality of patients as illustrated in FIG. 21, and may easily check messages exchanged with the patients as illustrated in FIG. 22.

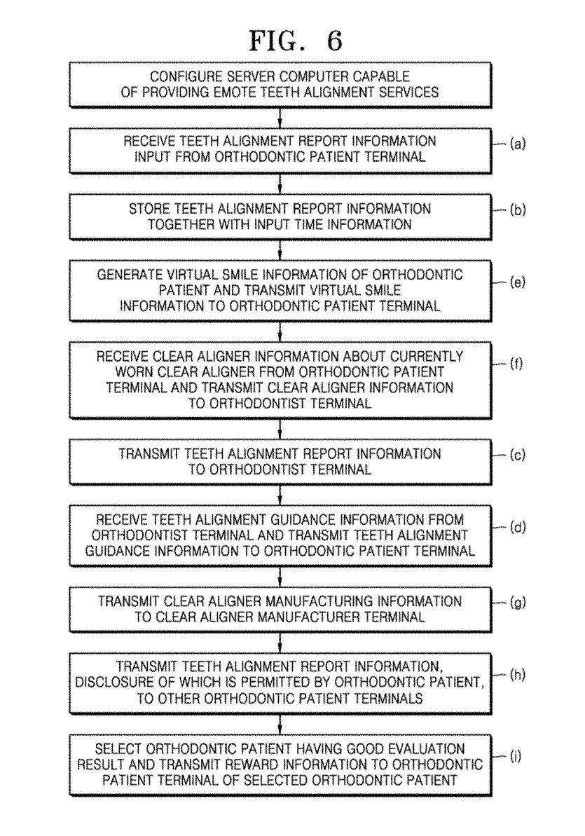

FIG. 6 is a detailed flowchart of a method of providing remote teeth alignment services, according to other embodiments of the present invention.

As illustrated in FIGS. 1 to 6, the remote teeth alignment service providing method according to other embodiments of the present invention may include configuring the above-described server computer 60, (a) receiving the teeth alignment report information, including photographs or videos, inputted from the orthodontic patient terminal 10 using the teeth alignment report information input program 64, (b) storing the inputted teeth alignment report information together with the input time information using the teeth alignment report information storage program 65, (e) generating the virtual smile information of the orthodontic patient based on the teeth alignment report information and transmitting the virtual smile information to the orthodontic patient terminal 10 or the orthodontist terminal 20 using the smile information transmission program 68, (f) receiving the clear aligner information about a currently worn clear aligner from the orthodontic patient terminal 10 and transmitting the clear aligner information to the orthodontist terminal 20 using the clear aligner information transmission program 69, (c) transmitting the teeth alignment report information stored together with the input time information, to the orthodontist terminal 20 using the teeth alignment report information transmission program 66, (d) receiving the teeth alignment guidance information from the orthodontist terminal 20 and transmitting the teeth alignment guidance information to the orthodontic patient terminal 10 using the teeth alignment guidance information transmission program 67, (g) transmitting the clear aligner manufacturing information to the clear aligner manufacturer terminal 30 upon determining that an additional or modified clear aligner is needed, based on the teeth alignment report information using the clear aligner manufacturing program 70, (h) transmitting the teeth alignment report information, disclosure of which is permitted by the orthodontic patient, to the other orthodontic patient terminals 10 using the information disclosure program 71, and (i) generating the evaluation result information about the teeth alignment report information, selecting an orthodontic patient having a good evaluation result based on the evaluation result information, and transmitting the reward information to the orthodontic patient terminal 10 of the selected orthodontic patient using the evaluation program 72.

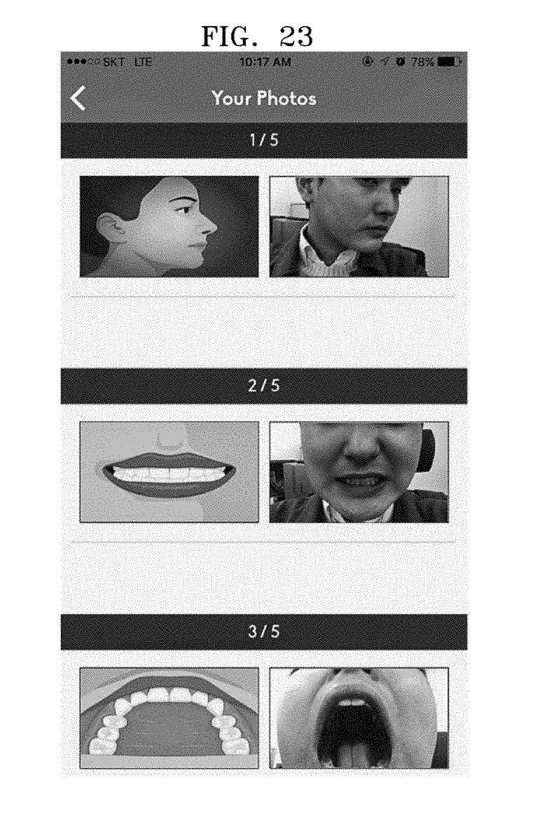

Herein, for example, in (e) the generating of the virtual smile information of the orthodontic patient based on the teeth alignment report information and the transmitting of the virtual smile information to the orthodontic patient terminal 10 or the orthodontist terminal 20, if the orthodontic patient inputs his/her face and teeth photos as illustrated in FIG. 23, a change in lips between before and after teeth alignment based on an aesthetic line may be viewed using a virtual photo.

Herein, for example, the aesthetic line may be a virtual line obtained by connecting a first skin point located in front of a nasion from which nasal bones start based on a profile skull line of a person, to a second skin point located in front of a pogonion. However, the aesthetic line is not limited thereto and may use one of various aesthetic reference lines for teeth alignment.

According to the afore-described embodiments of the present invention, an orthodontist may obtain accurate teeth alignment information using a device, e.g., a smartphone, of an orthodontic patient, may remotely and accurately diagnose the condition of the patient based on the teeth alignment information, and may remotely provide information about an accurate step of a clear aligner to be currently worn by the orthodontic patient, thereby greatly reducing a diagnosis time and cost of the orthodontic patient and the orthodontist. The accuracy of diagnosis may be improved by automatically obtaining photos of accurate sizes and locations using various virtual lines. The teeth alignment information may be easily used by the orthodontist by providing the teeth alignment information in the form of report information and classifying the teeth alignment information based on time. Teeth alignment may be precisely performed in a customized manner by providing manufacturing information to a clear aligner manufacturer when an additional or modified clear aligner is necessary. Interest of orthodontic patients may be increased by disclosing the teeth alignment information of a selected orthodontic patient, and the orthodontic patients may be inspired with a greater desire for aligned teeth by providing benefits to the selected orthodontic patient. As such, an operator may receive fees and charges from the orthodontic patient, the orthodontist, and the clear aligner manufacturer to provide remote teeth alignment services. The orthodontic patient, the orthodontist, and the clear aligner manufacturer may receive high-quality remote teeth alignment services, may greatly reduce times and costs required for teeth alignment, diagnosis, and clear aligner manufacturing, and clear aligner prices to be paid, and may easily receive additional or modified clear aligners and various types of information and convenience. Therefore, advantages may be provided to all. However, the scope of the present invention is not limited to the above effects.

The present invention can also be embodied as computer-readable code on a computer-readable medium.

The computer-readable medium is any data storage device that can store data which can be thereafter read by a computer system.

Examples of the computer-readable medium include not only the above-described server computer 60 but also read-only memory (ROM), random-access memory (RAM), CD-ROMs, magnetic tapes, floppy disks, magneto-optical disks, optical data storage devices, flash memory, and carrier waves (e.g., transmission through the Internet).

The computer-readable medium can also be distributed over network coupled computer systems so that the computer-readable code is stored and executed in a distributed fashion.

While the present invention has been particularly shown and described with reference to embodiments thereof, it will be understood by one of ordinary skill in the art that various changes in form and details may be made therein without departing from the spirit and scope of the present invention as defined by the following claims.

What is claimed is:

1. A method of providing remote teeth alignment services using a computer system comprising at least one orthodontic patient terminal, an orthodontist terminal, and a server computer connected to a network, the method comprising:
receiving a teeth alignment report information inputted from the orthodontic patient terminal;
storing the received teeth alignment report information; and
transmitting the stored teeth alignment report information to the orthodontist terminal.

2. A method of providing remote teeth alignment services using a computer system comprising at least one orthodontic patient terminal, an orthodontist terminal, and a server computer connected to a network, the server computer comprising:
a teeth alignment report information input program for receiving a teeth alignment report information inputted from the orthodontic patient terminal;
a teeth alignment report information storage program for storing the inputted teeth alignment report information together with an input time information;
a teeth alignment report information transmission program for transmitting the teeth alignment report information stored together with the input time information, to the orthodontist terminal; and
a teeth alignment report information database for storing the teeth alignment report information, and
the method comprising:
(a) receiving the teeth alignment report information, including photographs or videos, inputted from the orthodontic patient terminal using the teeth alignment report information input program;
(b) storing the inputted teeth alignment report information together with the input time information using the teeth alignment report information storage program; and
(c) transmitting the teeth alignment report information stored together with the input time information, to the orthodontist terminal using the teeth alignment report information transmission program.

3. The method of claim 2,
wherein the teeth alignment report information comprises at least one of photographing step information, photographing date information, and a front-view teeth photo, a bottom-view low-angle teeth photo, a front-view automatically-recognized mouth gag photo, a front-view automatically-recognized face contour photo, a left-side-angle automatically-recognized mouth gag photo, a left-side-angle automatically-recognized face contour photo, a right-side-angle automatically-recognized mouth gag photo, a right-side-angle automatically-recognized face contour photo, a collection of top, bottom, left, and right side photos surrounding a central front photo, a low-angle upper jaw photo, a high-angle lower jaw photo, and combinations thereof, which are taken using a camera of the orthodontic patient terminal.

4. The method of claim 2,
wherein the teeth alignment report information input program comprises at least one of:
a camera activation program for activating a camera of the orthodontic patient terminal;
a current step information input program;
a current date information input program;
a text guidance information display program for displaying text guidance information to an orthodontic patient;
a voice guidance information output program for outputting voice guidance information to the orthodontic patient;
a face contour automatic-recognition photographing program for displaying a virtual face contour line on a screen of the orthodontic patient terminal and automatically recognizing and taking a photo of a face contour of the orthodontic patient shown on the screen;
a teeth contour automatic-recognition photographing program for displaying a virtual teeth line on the screen of the orthodontic patient terminal and automatically recognizing and taking a photo of teeth of the orthodontic patient shown on the screen;
a mouth gag contour automatic-recognition photographing program for displaying a virtual mouth gag contour line on the screen of the orthodontic patient terminal and automatically recognizing and taking a photo of a mouth gag shown on the screen;
a photo check program for displaying the taken photos on the screen; and
combinations thereof.

5. The method of claim 2,
wherein the server computer further comprises:
a teeth alignment guidance information transmission program for receiving a teeth alignment guidance information from the orthodontist terminal and transmitting the teeth alignment guidance information to the orthodontic patient terminal; and
a teeth alignment guidance information database for storing the teeth alignment guidance information, and
wherein the method further comprises:
(d) receiving the teeth alignment guidance information from the orthodontist terminal and transmitting the teeth alignment guidance information to the orthodontic patient terminal using the teeth alignment guidance information transmission program, after step (c).

6. The method of claim 5,
wherein the teeth alignment guidance information comprises at least one of:
a clear aligner information about a clear aligner to be worn in a current step among a clear aligner set;
a wearing time information about a wearing time of the clear aligner;
a replacement information about replacement of a part of or the whole clear aligner set;
a clear aligner state information about the state of the clear aligner;
a clear aligner usage guidance information;
a question information;
a memo information;
a clear aligner precautions information;
a dental clinic visit information; and
combinations thereof.

7. The method of claim 2,
wherein the server computer further comprises:
a smile information transmission program for generating a virtual smile information of an orthodontic patient based on the teeth alignment report information and transmitting the virtual smile information to the orthodontic patient terminal; and
a smile information database for storing the virtual smile information, and
wherein the method further comprises:
(e) generating the virtual smile information of the orthodontic patient based on the teeth alignment report information and transmitting the virtual smile information to the orthodontic patient terminal or the orthodontist terminal using the smile information transmission program, after step (b).

8. The method of claim 2,
wherein the server computer further comprises:
a clear aligner information transmission program for receiving a clear aligner information about a currently worn clear aligner from the orthodontic patient terminal, storing the clear aligner information, and transmitting the clear aligner information to the orthodontist terminal; and
a clear aligner information database for storing the clear aligner information, and
wherein the method further comprises:
(f) receiving the clear aligner information about a currently worn clear aligner from the orthodontic patient terminal and transmitting the clear aligner information to the orthodontist terminal using the clear aligner information transmission program, after step (b).

9. The method of claim 2,
wherein the teeth alignment report information transmission program comprises at least one of:
a patient-specific information transmission program for transmitting a teeth alignment information per orthodontic patient;
a current step information transmission program for transmitting a current clear aligner step information;
a photographing schedule transmission program for transmitting a photographing schedule information;
a schedule adjustment information transmission program for adjusting the photographing schedule of the orthodontic patient;
a calendar information transmission program for transmitting a calendar information per orthodontic patient;
an individual memo transmission program for transmitting a memo information per orthodontic patient;
an integrated memo information transmission program for transmitting a memo information to all orthodontic patients; and
combinations thereof.

10. The method of claim 2,
wherein the server computer further comprises:
a clear aligner manufacturing program for transmitting a clear aligner manufacturing information to a clear aligner manufacturer terminal upon determining that an additional or modified clear aligner is needed, based on the teeth alignment report information; and
a clear aligner manufacturing information database for storing the clear aligner manufacturing information, and
wherein the method further comprises:
(g) transmitting the clear aligner manufacturing information to the clear aligner manufacturer terminal upon determining that an additional or modified clear aligner is needed, based on the teeth alignment report information using the clear aligner manufacturing program, after step (c).

11. The method of claim 2,
wherein the server computer further comprises an information disclosure program for transmitting the teeth alignment report information, disclosure of which is permitted by an orthodontic patient, to other orthodontic patient terminals, and
wherein the method further comprises:
(h) transmitting the teeth alignment report information, disclosure of which is permitted by the orthodontic patient, to the other orthodontic patient terminals using the information disclosure program, after step (c).

12. The method of claim 2,
wherein the server computer further comprises:
an evaluation program for generating an evaluation result information about the teeth alignment report information, selecting an orthodontic patient having a good evaluation result based on the evaluation result information, and transmitting a reward information to the orthodontic patient terminal of the selected orthodontic patient; and
an evaluation result information database for storing the evaluation result information, and
wherein the method further comprises:
(i) generating the evaluation result information about the teeth alignment report information, selecting an orthodontic patient having a good evaluation result based on the evaluation result information, and transmitting the reward information to the orthodontic patient terminal of the selected orthodontic patient using the evaluation program, after step (c).

13. A computer system for providing remote teeth alignment services using at least one orthodontic patient terminal, an orthodontist terminal, and a server computer connected to a network, the server computer comprising:
a teeth alignment report information input program for receiving a teeth alignment report information inputted from the orthodontic patient terminal;
a teeth alignment report information storage program for storing the input teeth alignment report information together with an input time information;
a teeth alignment report information transmission program for transmitting the teeth alignment report information stored together with the input time information, to the orthodontist terminal;
a teeth alignment report information database for storing the teeth alignment report information; and
a controller programmed to receive the teeth alignment report information, including photographs or videos, inputted from the orthodontic patient terminal using the teeth alignment report information input program, to store the input teeth alignment report information together with the input time information using the teeth alignment report information storage program, and to transmit the teeth alignment report information stored together with the input time information, to the orthodontist terminal using the teeth alignment report information transmission program.

14. A computer-readable medium on which is recorded a method of providing remote teeth alignment services using a computer system comprising at least one orthodontic patient terminal, an orthodontist terminal, and a server computer connected to a network, the server computer comprising:
a teeth alignment report information input program for receiving a teeth alignment report information inputted from the orthodontic patient terminal;
a teeth alignment report information storage program for storing the input teeth alignment report information together with an input time information;
a teeth alignment report information transmission program for transmitting the teeth alignment report information stored together with the input time information, to the orthodontist terminal; and
a teeth alignment report information database for storing the teeth alignment report information, and
the method comprising:
(a) receiving the teeth alignment report information, including photographs or videos, inputted from the orthodontic patient terminal using the teeth alignment report information input program;
(b) storing the inputted teeth alignment report information together with the input time information using the teeth alignment report information storage program; and
(c) transmitting the teeth alignment report information stored together with the input time information, to the orthodontist terminal using the teeth alignment report information transmission program.
